SEQUENCE LISTING (3 sequences)

SEQ ID NO: 1 - DNA, 7 bp, Artificial Sequence
  Description of Artificial Sequence Synthetic oligonucleotide
gaacaag

SEQ ID NO: 2 - RNA, 13 nt, Artificial Sequence
  Description of Artificial Sequence Synthetic oligonucleotide, mammalian Kozak sequence
gccgccaccaugg

SEQ ID NO: 3 - RNA, 15 nt, Artificial Sequence
  Description of Artificial Sequence Synthetic oligonucleotide, stabilizing sequence
yccancccwyucycc

(19) United States
(12) Patent Application Publication
(10) Pub. No.: US 2012/0009221 A1
(43) Pub. Date: Jan. 12, 2012

(54) TRANSFECTION OF BLOOD CELLS WITH MRNA FOR IMMUNE STIMULATION AND GENE THERAPY

(75) Inventors: INGMAR HOERR, TUEBINGEN (DE); STEVE PASCOLO, TUEBINGEN (DE); FLORIAN VAN DER MÜLBE, TUEBINGEN (DE)

(73) Assignee: CUREVAC GMBH

(21) Appl. No.: 13/174,081

(22) Filed: Jun. 30, 2011

Related U.S. Application Data
(63) Continuation of application No. 11/342,392, filed on Jan. 30, 2006, now abandoned.
Application No. 11/342,392 is a continuation of application No. PCT/EP2004/008459, filed on Jul. 28, 2004.

(30) Foreign Application Priority Data
Aug. 5, 2003 (DE) 103 35 833.1

(51) Int. Cl. A61K 39/00; A61P 37/04; A61P 31/00; C12N 15/85; A61P 35/00

(52) U.S. Cl. 424/277.1; 424/184.1; 435/455

(57) ABSTRACT

The present invention relates to a pharmaceutical composition containing blood cells or haemopoietic cells, e.g. red blood cells (erythrocytes), granulocytes, mononuclear cells (PBMCs) and/or blood platelets, in combination with a pharmaceutically acceptable excipient and/or vehicle, wherein the cells are transfected with at least one mRNA comprising at least one region coding for at least one antigen. The invention further discloses a method of preparing the aforesaid pharmaceutical composition and the use of blood cells transfected in this way for the preparation of drugs or pharmaceutical compositions for immune stimulation against the antigens encoded by the mRNA. The subjects according to the invention are used especially for the therapy and/or prophylaxis of carcinoses or infectious diseases and can also be employed in gene therapy.

### RELATED APPLICATIONS

The present application is a Continuation of cow pending PCT Application No, PCT/EP2004/008459, filed Jul. 2, 2004 which in turn, claims priority from German Application Serial No. 103 35 833.1, filed Aug. 5, 2003. Applicants claim the benefits of 35 U.S.C. §120 as to the PCT application and priority under 35 U.S.C. §119 as to said. German application, and the entire disclosures of both applications are incorporated herein by reference in their entireties.

### BACKGROUND OF THE INVENTION

The present invention relates to a pharmaceutical composition containing blood cells or haemopoietic cells, e.g. red blood cells (erythrocytes), granulocytes, mononuclear cells (PBMCs) and/or blood platelets, in combination with a pharmaceutically acceptable excipient and/or vehicle, wherein the cells are transfected with at least one mRNA comprising at least one region coding for at least one antigen. The invention further discloses a method of preparing the aforesaid pharmaceutical composition and the use of blood cells transfected in this way for the preparation of medicaments or pharmaceutical compositions for immune stimulation against the antigens encoded by the mRNA. The subjects according to the invention are used especially for the therapy and/or prophylaxis of carcinoses or infectious diseases and can also be employed in gene therapy.

Gene therapy and genetic vaccination are procedures of molecular medicine whose application in the therapy and prevention of diseases will have considerable effects on medical practice. Both procedures are based on the introduction of nucleic acids into the patient's cells or tissues and on subsequent processing of the information encoded by the nucleic acids introduced, i.e. the expression of the desired polypeptides. In principle, advantageous procedures are based on the direct introduction or indirect introduction (via transfection of suitable cells) of (m)RNA coding for the particular protein.

At the present time in the use of mRNA for vaccination, especially vaccination against tumour antigens, the mRNA coding for the appropriate antigen is conventionally transfected in vitro into (professional) antigen presenting cells (APCs), especially dendritic cells (DCs) (Boczkowski et al. (1996) J. Exp. Med, 184(2), 465-472; WO 97/41210). The DCs are obtained from the patient's blood by differentiating appropriate monocytes into DCs using a special cytokine cocktail. However, this is a lengthy procedure. Specifically, the period between blood withdrawal and transfection is conventionally about 7 days (i.e. about 1 week of in vitro culture for differentiation of the DCs), so it generally takes 9 days (2 days of maturation of the DCs after transfection) before the cells are injected back into the patient. The decisive factor in this long period is the duration of the in vitro culture for differentiation of the DCs between blood withdrawal and transfection. Furthermore, GMP (good manufacturing practice) conditions have to be observed when producing DCs. This procedure is therefore extremely costly and it also has to be borne in mind that the patient may require special accommodation during DC production.

The object of the present invention is therefore to provide a novel system for the immune stimulation of patients against specific antigens, especially antigens from tumours and infectious germs, which overcomes the disadvantages of procedures known in the state of the art and, in particular, avoids a laborious and expensive production of APCs in vitro.

This object is achieved by the embodiments of the present invention characterized in the Claims.

### SUMMARY OF TEE INVENTION

In particular, according to the invention, a pharmaceutical composition is provided which contains blood cells or haemopoietic cells, and particularly whole blood cells that have not been treated or otherwise cultivated, etc., that are transfected with at least one mRNA, e.g. red blood cells (erythrocytes), granulocytes, mononuclear cells (peripheral blood mononuclear cells, PBMCs) and/or blood platelets (thrombocytes), in combination with a pharmaceutically acceptable excipient and/or vehicle, wherein the at least one mRMA comprises at least one region coding for at least one antigen.

The present invention is based on the surprising discovery that, to vaccinate patients against certain antigens encoded by the mRNA according to the invention, it is not necessary to differentiate blood cells, e.g. PBMCs, DCs, etc. obtained e.g. from the blood of an individual, especially the actual patient to be treated, by means of laborious, lengthy and expensive cell culture techniques, into a population of cells with a high proportion of professional antigen presenting cells (APCs), especially dendritic cells (DCs), but that it is sufficient, for a successful immune stimulation, to transfect such untreated blood cells directly with the mRNA coding for one or more antigens in order to obtain a pharmaceutical composition which effects a suitable immune stimulation e.g. in the actual patient from whom the blood cells, especially the abovementioned partial populations thereof, have been obtained, said immune stimulation preferably being directed against one or more antigens from a tumour or one or more antigens from a pathogenic germ or agent.

The blood cells, especially the abovementioned partial populations thereof, in the pharmaceutical composition according to the invention are characterized in particular in that they contain a small proportion of well-differentiated professional APCs, such as DCs. The transfected cells contain preferably less than 5%, particularly preferably no more than 2%, of DCs when they are contained in the pharmaceutical composition of the present invention.

“Blood cells” are therefore preferably understood according to the invention as meaning a mixture or an enriched to substantially pure population of red blood cells, granulocytes, mononuclear cells (PBMCs) and/or blood platelets from whole blood, blood serum or another source, e.g. from the spleen or lymph nodes, only a small proportion of professional APCs being present. In contrast to the state of the art, the blood cells of the present invention are preferably fresh blood cells, i.e. the period between collection of the blood cells (especially blood withdrawal) and transfection being only short, e.g. less than 12 h, preferably less than 6 h, particularly preferably less than 2 h and very particularly preferably less than 1 h. As such, the term “blood cells” as defined and used herein, also means “untreated blood cells” or “whole blood cells”.

As already mentioned above, the blood cells used for the pharmaceutical composition according to the invention preferably originate from the actual patient who will be treated with the pharmaceutical composition of the present invention. The pharmaceutical composition according to the invention therefore preferably contains or consists essentially of, autologous blood cells.

In another preferred embodiment of the present invention, the mRNA used for transfection of the cells according to the invention comprises a region coding for at least one antigen from a tumour, a pathogenic agent or a pathogenic germ.

According to the invention, the expression “antigen from a tumour” means that the appropriate antigen is expressed in cells associated with a tumour. Therefore, according to the invention, antigens from tumours are especially those produced in the degenerate cells themselves. They are preferably antigens located on the surface of the cells. However, the antigens from tumours are also antigens expressed in cells which are (were) not themselves (or not originally themselves) degenerate, but which are associated with the tumour in question. These also include e.g. antigens associated with tumour supply vessels or their (re)generation, especially antigens associated with neovascularization or angiogenesis, e.g. growth factors such as VEGF, bFGF, etc. Such antigens associated with a tumour also include those from cells of the tissue where the tumour is embedded. Appropriate antigens from connective tissue cells, e.g. antigens from the extracellular matrix, may be mentioned here. For the immune stimulation according to the invention, the mRNA molecules can also represent a cDNA library from a tumour tissue. It is also possible to use part of an appropriate library, preferably that part of the cDNA library which codes for the tumour-specific antigens. Corresponding methods for preparation will be described in detail below in connection with the method according to the invention for the preparation of the pharmaceutical composition.

According to the invention, the pharmaceutical composition containing blood cells, e.g. PBMCs, red blood cells, granulocytes and/or blood platelets, transfected with one or more mRNAs is used for therapy or inoculation, i.e. vaccination, especially for the treatment or prevention (prophylaxis) of carcinoses. Vaccination is based on the introduction of an antigen or several antigens) from a tumour, in the present case the genetic information for the antigen in the form of the mRNA coding for the antigen(s), into the blood cells. The mRNA contained in the pharmaceutical composition is translated into the (tumour) antigen in the cells, i.e. the polypeptide or antigenic peptide encoded by the modified mRNA is expressed, whereby, after introduction of the pharmaceutical composition containing the transfected cells, an immune response directed against this polypeptide or antigenic peptide is stimulated. In the present case of use as a genetic vaccine for the treatment of cancer, the immune response is therefore achieved by introducing the genetic information for antigens from a tumour, especially proteins expressed exclusively on cancer cells, by administering a pharmaceutical composition according to the invention that contains blood cells, e.g. red blood cells, granulocytes, PBMCs and/or blood platelets, transfected with an mRNA coding for such a cancer antigen. The cancer antigen(s) is (are) thereby presented to immune cells in the organism, which causes an immune response directed effectively against the cancer cells.

In the case of vaccination against a pathogenic germ such as a virus, a bacterium or a protozoological germ, it is therefore preferable to use a surface antigen from such a germ in order to vaccinate with the aid of the pharmaceutical composition according to the invention that contains blood cells transfected with the mRNA coding for the surface antigen. The pharmaceutical composition according to the invention is therefore also used particularly against infectious diseases (e.g. viral infectious diseases such as AIDS (HIV), hepatitis A, B or C, herpes, herpes zoster (varicella), German measles (rubella virus), yellow fever, dengue, etc. (flaviviruses), influenza (influenza viruses), haemorrhagic infectious diseases (Marburg or Ebola viruses), bacterial infectious diseases such as legionnaire's disease (Legionella), gastric ulcer (Helicobacter), cholera (Vibrio), B. coli infections, Staphylococcus infections, Salmonella infections or Streptococcus infections (tetanus), or protozoological infectious diseases (malaria, sleeping sickness, leishmaniasis, toxoplasmosis, i.e. infections due to Plasmodium, trypanosomes, Leishmania and Toxoplasma, as well as Chlamydia infections, e.g. with Chlamydia pneumoniae or Chlamydia trachomatis)). Preferably, in the case of infectious diseases, the appropriate surface antigens with the strongest antigenic potential are also encoded by the mRNA. In the case of said genes of pathogenic germs or organisms, especially viral genes, this is typically a secreted form of a surface antigen.

In the case of immune stimulation against a pathogenic germ, the transfected mRNA molecules can also comprise a cDNA library, or part thereof, of certain cell types. In this case it is preferable according to the invention to use cDNA (partial) libraries of cells infected with the pathogenic agent. Thus, in the case of an HIV infection, for example, it is possible to use an mRNA population from HIV-infected, preferably autologous T cells in order to achieve immune stimulation against the products of the host genes that are upregulated by the pathogenic agent (HIV in the example given).

It is also preferable according to the invention to use mRNAs coding for polypeptides, the polypeptides being polyepitopes, e.g. the abovementioned antigens, especially surface antigens from pathogenic gems/organisms or tumour cells, and preferably secreted forms of proteins.

In its use as a vaccine, the pharmaceutical composition according to the invention is particularly suitable for the treatment of carcinoses (the mRNA preferably coding for a tumour-specific surface antigen (TSSA)), e.g. the treatment of malignant melanoma, colon carcinoma, lymphomas, sarcomas, small cell pulmonary carcinoma, blastomas, etc. Specific examples of tumour antigens are, inter alia, 707-AP, AFP, ART-4, BAGS, β-catenin/m, Bcr-abl, CAMEL, CAP-1, CASP-8, CDC27/m, CDK4/m, CEA, CT, Cyp-R, DAM, ELF2M, ETV6-AML1, G250, GAGE, GnT-V, Gp100, HAGE, HER-2/neu, HLA-A*0201-R170I, HPV-E7, HSP70-2M, HAST-2, hTERT or hTRT), iCE, KIAA0205, LAGE, LDLR/FUT, MAGE, MART-1/melan-A, MC1R, myosin/m, MUC1, MUM-1, -2, -3, NA88-A, NY-ESO-1, p190 minor bcr-abl, PmI/RARα, PRAME, PSA, PSM, RAGE, RU1 or RU2, SAGE, SART-1 or SART-3, TEL/AML1, TPI/m, TRP-1, TRP-2, TRP-2/INT2 and WT1.

In another preferred embodiment, the antigen(s) from a tumour or a pathogenic agent is (are) a polyepitope of the antigen(s) from a tumour or a pathogenic agent. A “polyepitope” of an antigen or several antigens is an amino acid sequence in which several or many regions of the antigen(s) are represented, said regions interacting with the antigen-binding part of an antibody or with a T cell receptor. The polyepitope can be in a complete and unmodified form, but, according to the present invention, it can also be in a modified form, especially for optimising the antibody/antigen or T cell receptor/antigen interaction. A modification relative to the wild-type polyepitope can include e.g. a deletion, addition and/or substitution of one or more amino acid residues.

Accordingly, one or more nucleotides are deleted, added and/or substituted in the mRNA of the present invention that codes for the modified polyepitope, relative to the mRNA coding for the wild-type polyepitope.

Furthermore, the pharmaceutical composition according to the invention can be used in gene therapy, the RNA coding e.g. for a protein, for example an enzyme, that is missing or not functioning in a patient's blood cells or haemopoietic cells.

To increase the stability and transfection efficiency of the (m)RNA, each (m)RNA to be introduced into the blood cells of the present invention preferably has one or more modifications, especially chemical modifications, which improve the transfer of the (m)RNA(s) into the cells to be transfected and/or increase the expression of the encoded antigen(s).

For example, the sequences of eukaryotic mRNAs contain destabilizing sequence elements (DSEs) to which signal proteins bind and regulate the enzymatic degradation of the mRNA in vivo. Therefore, for further stabilization of the mRNA, one or more changes are optionally made in the region coding for the at least one antigen from a tumour or pathogenic agent, relative to the corresponding region of the wild-type mRNA, so that no destabilizing sequence elements are present. Of course, it is also preferred according to the invention to eliminate from the mRNA any DSEs present in the untranslated regions (3′- and/or 5′-UTR).

Examples of such destabilizing sequences are AU-rich sequences (“AU-RES”), which occur in 3′-UTR segments of numerous unstable RNAs (Caput at al., Proc. Natl. Acad. Sci. USA 1986, 83, 1670 to 1674). The RNA molecules used in the present invention are therefore preferably changed, relative to the wild-type mRNA, in such a way that they do not have any such destabilizing sequences. This also applies to sequence units (motifs) recognized by possible endonucleases, e.g. the sequence GAACAAG contained in the 3′ UTR segment of the gene coding for the transferrin receptor (Binder at al., EMBO J. 1994, 13, 1969 to 1980). These sequence units (motifs) are also preferably eliminated from the modified mRNA used for transfection of the blood cells.

Those skilled in the art are familiar with various methods that are suitable for substituting codons in the mRNA modified according to the invention. In the case of shorter coding regions (coding for biologically effective or antigenic peptides), it is possible, for example, to synthesize the total mRNA chemically using standard techniques.

Preferably, however, base substitutions are introduced using a DNA template in order to prepare the modified mRNA by common directed mutagenesis techniques (Maniatis at al., Molecular Cloning; A Laboratory Manual, Cold Spring Harbor Laboratory Press, 3rd ed., Cold Spring Harbor, N.Y., 2001).

In this method, therefore, an appropriate DNA molecule is transcribed in vitro in order to prepare the mRNA. This DNA template has a suitable promoter, e.g. a T7 or SP6 promoter, for the in vitro transcription, which is followed by the desired nucleotide sequence for the mRNA to be prepared and by a termination signal for the in vitro transcription. According to the invention, the DNA molecule that forms the template of the RNA construct to be prepared is conventionally prepared by fermentative multiplication and subsequent isolation as part of a plasmid replicatable in bacteria. Examples which may be mentioned of plasmids suitable for the present invention are pT7TS (GenBank Accession Number U26404; Lai et al., Development 1995, 121, 2349 to 2360), the pGEM® series, e.g. pGEM®-1 (GenBank Accession Number X65300; from Promega), and pSP64 (GenBank Accession Number X65327) (cf. also Mezei and Storts, Purification of PCR Products, in Griffin and Griffin (eds), PCR Technology: Current Innovation, CRC Press, Boca Raton, Fla., 2001).

Thus the desired nucleotide sequence can be cloned into a suitable plasmid by molecular biological methods familiar to those skilled in the art using short synthetic DNA oligonucleotides which have short single-stranded transitions at the existing restriction sites, or using genes prepared by chemical synthesis (cf. Maniatis et al., op. cit.). The DNA molecule is then cleaved from the plasmid, in which it can be present in single or multiple copy, by digestion with restriction endonucleases.

The modified mRNA which can be used for transfection of the blood cells can also have a 5′ cap structure (a modified guanosine nucleotide). Examples of cap structures which may be mentioned are m7G(5′)ppp, (5′)A,G(5′)ppp(5′)A and G(5′)ppp(5′)G.

In another preferred embodiment of the present invention, the modified mRNA contains a poly(A⁺) tail of at least about 25, especially of at least about 30, preferably of at least about 50, particularly preferably of at least about 70 and very particularly preferably of at least about 100 nucleotides. However, the poly(A⁺) tail can also comprise 200 nucleotides or more.

Efficient translation of the mRNA further requires an effective binding of the ribosomes to the ribosome binding site (Kozak sequence: GCCGCCACCAUGG, the AUG forming the start codon). It has been found in this regard that an increased A/U content around this site enables a more efficient ribosome binding to the mRNA.

It is also possible to insert one or more so-called IRESs (internal ribosomal entry sites) into the mRNA. An IRES can thus act as a single ribosome binding site but it can also be used to provide an mRNA coding for several (e.g. two) peptides or polypeptides which are to be translated independently of one another by the ribosomes in the PEMCs (“multicistronic” or “polycistronic” (e.g. bicistronic) mRNA). Examples of IEEE sequences which can be used according to the invention are those from picornaviruses (e.g. FMDV), plague viruses (CFPV), polioviruses (PV), encephalomyocarditis viruses (ECMV), foot-and-mouth disease viruses (FMDV), hepatitis C viruses (HCV), classical swine fever viruses (CSFV), murine leukora virus (MLV), simean immunodeficiency viruses (SIV) or cricket paralysis viruses (CrPV).

In another preferred embodiment of the present invention, in the 5′- and/or 3′-untranslated regions, the mRNA has stabilizing sequences capable of increasing the half-life of the mRNA in the cytosol.

These stabilizing sequences can have a 100% sequence homology to naturally occurring sequences that appear in viruses, bacteria and eukaryotes, but they can also be of a partially or completely synthetic nature. Examples which may be mentioned of stabilizing sequences that can be used in the present invention are the untranslated sequences (UTR) of the α and β-globin gene, e.g. from Homo sapiens or Xenopus laevis. Another example of a stabilizing sequence has the general formula (C/U)CCANₓCCC(U/A)PyₓUC(C/U)CC, which is present in the 3′-UTR of the very stable mRNA coding for α-globin, α-(1)-collagen, 15-lipoxygenase or tyrosine hydroxylase (cf. Holcik et al., Proc. Nati. Asad. Sci. USA 1997, 94, 2410 to 2414). Of course, such stabilizing sequences can be used individually, in combination with one another or in combination with, other stabilizing sequences known to those skilled in the art.

For further stabilization, the mRNA also preferably has at least one analogue of naturally occurring nucleotides. This is based on the fact that the RNA-degrading enzymes occurring in the blood cells preferentially recognize naturally occurring nucleotides as substrate. The RNA degradation can therefore be made more difficult by inserting nucleotide analogues, it being possible for the insertion of these analogues, especially into the coding region of the mRNA, to have a positive or negative effect on the translation efficiency.

The following may be mentioned as examples of nucleotide analogues that can be used according to the invention, without the list in any way being definitive phosphoramidates, phosphorothioates, peptide nucleotides, methylphosphonates, 7-deazaguanosine, 5-methylcytosine and inosine. The preparation of such analogues is known to those skilled in the art e.g. from patents U.S. Pat. No. 4,373,071, U.S. Pat. No. 4,401,796, U.S. Pat. No. 4,415,732, U.S. Pat. No. 4,458,066, U.S. Pat. No. 4,500,707, U.S. Pat. No. 4,668,777, U.S. Pat. No. 4,973,679, U.S. Pat. No. 5,047,524, U.S. Pat. No. 5,132,418, U.S. Pat. No. 5,153,319, U.S. Pat. No. 5,262,530 and U.S. Pat. No. 5,700,642. According to the invention, such analogues can occur in untranslated and translated regions of the modified mRNA.

Furthermore, the effective transfer of the preferably modified mRNA into the cells can be improved if the mRNA is associated with or bound to a cationic or polycationic agent, especially an appropriate peptide or protein, prior to transfection of the previously obtained blood cells. The mRNA is therefore preferably complexed or condensed with such an agent prior to transfection of the PEMCs. It is particularly effective here to use protamine as a polycationic, nucleic acid-binding protein. It is also possible to use other cationic peptides or proteins, such as poly-L-lysine, poly-L-arginine or histones. This procedure for stabilizing the modified mRNA is described in EP-A-1083232, whose relevant content of disclosure is fully included in the present invention. The mRNA for transfection into the cells can also be associated or mixed with other substances for efficient transfer. Examples of this are inclusion in microparticles or nanoparticles, especially those based on PLGA (poly(D,L-lactide-co-glycolide)), and lipids.

Furthermore, according to the invention, the mRNA can also contain, in addition to the antigenic peptide/polypeptide or the peptide/polypeptide effective for gene therapy, at least one other functional segment coding e.g. for a cytokine that promotes the immune response (monokine, lymphokine, interleukin or chemokine, such as IL-1, IL-2, IL-3, IL-4, IL-5, IL-6, IL-7, IL-8, Its-9, IL-10, IL-12, IFN-α, IFN-γ, GM-CSF and LT-α) or growth factors such as hGH. Alternatively or additionally, the mRNA provided for transfection of the blood cells or haemopoietic cells, especially red blood cells, PBMCs, granulocytes and/or blood platelets, can also code for at least one co-stimulating molecule (e.g. CD40, CD80, CD86 or 4-1BB ligand) and/or at least one transcription factor (e.g. NF-kappaB or ICSBP (interferon consensus binding protein)), which assures a particularly efficient expression of immunostimulating molecules in the transfected cells, and/or for at least one homing receptor (e.g. CCR7), which directs the transfected cells e.g. into the lymph nodes, and/or for at least one suicide molecule (e.g. herpes simplex virus thymidine kinase (HSV-tk), cytochrome P450 4B1 (cyp4B1) and/or folylpolyglutamate synthase (FPGS)), which is expressed in the transfected cells and converts an otherwise inactive prodrug to its active form (e.g. nucleoside analogues, such as ganciclovir or acyclovir, by HSV-tk, and/or 4-ipomeanol or 2-aminoanthracene by cyp4B1), or intensifies the action of a chemotherapeutic agent that is already effective per se alkylating agents, such as methotrexate, by FPGS), and thereby induces necrotic and/or apoptotic cell death, resulting in the release of the cell contents, which include the antigen encoded by the mRNA.

The present patent application also provides a method of preparing the pharmaceutical composition defined above, comprising the following steps:
(a) collection of blood cells, and
(b) transfection of the blood cells in vitro with at least one mRNA comprising at least one region coding for at least one antigen.

Blood cells are collected preferably directly, from an animal or human patient or host, by standard collection methods, for example. Thus whole blood can easily be obtained by puncturing a suitable vessel. Serum is obtained in a known manner by coagulating the solid blood constituents. PBMCs may be mentioned as an example of an enriched partial population of blood cells. These are conventionally isolated by a method first described by Bøyum (Nature 204, 793-794, 1964 Scan, J. Lab. Clin. Invest. Sunpl. 97, 1967). This is generally done by withdrawing blood from the individual and adding it e.g. to a solution of density 1,077 g/ml (25° C.), conventionally containing Ficoll and sodium diatrizoate, for density gradient centrifugation. During careful centrifugation at room temperature, the PBMCs collect at the Ficoll/blood interface whereas the red blood cells and the remaining white blood cells are sedimented. The interface with the PBMCs is recovered and conventionally washed with a suitable buffer, e.g. sterile PBS. The PBMCs are preferably subjected to a short isotonic treatment with an aqueous solution of e.g. ammonium chloride. Finally, the PBMCs are washed a further one or more times with a buffer such as PBS (sterile). The cells obtained can then optionally be stored under suitable conditions, conventionally at −70° C., until further use.

According to the invention, the blood cells immediately prior to transfection are preferably fresh blood cells, i.e. there is only a short period between the collection of blood cells (especially blood withdrawal) in step (a) and the transfection according to step (b), e.g. less than 12 h, preferably less than 6 h, particularly preferably less than 2 h and very particularly preferably less than 1 h. More particularly, such blood cells are untreated, as by known cultivation or differentiation procedures.

The transfection of the blood cells is likewise carried out by common methods, e.g. by means of electroporation or chemical methods, especially lipofection.

As regards preferred embodiments of the blood cells, reference is made to the above remarks relating to the pharmaceutical composition of the present invention. Similarly, as regards preferred embodiments of the mRNA in step (b) of the preparation method according to the invention, reference is made to the appropriate embodiments of the pharmaceutical composition according to the invention.

In another preferred embodiment, the transfected cells are subjected after step (b) to a step (c) for stimulation with immunostimulating agents such as LPS, TNF-α, Toll-like receptor ligands such as double-stranded RNA, stabilized DNA or CpG-DNA, etc.

As an example of blood cells that can be used according to the invention, PBMCs, as already described in detail above, can be obtained by Ficoll-Hypaque density gradient centrifugation, one or more subsequent washing steps with phosphate buffered saline (PBS) being carried out if necessary.

The mRNA for the transfection according to the invention is prepared by methods known to those skilled in the art, especially by chemical synthesis or particularly preferably by means of molecular biological methods which have already been mentioned above.

As already explained above, the mRNA for the in vitro transfection according to step (b) is complexed or condensed with at least one cationic or polycationic agent in order to increase the stability of the mRNA, which leads to a better transfection efficiency and, in particular, to increased expression rates in the transfected cells. Examples of suitable cationic or polycationic agents are protamine, poly-L-lysine, poly-L-arginine or histones (cf. in this respect the content of disclosure of EP-A-1083232 already cited above).

In one preferred embodiment, the above method of preparing the pharmaceutical composition defined above comprises the following steps:
(1) preparation of a cDNA library or part thereof from a patient's tumour tissue or from a patient's cells infected with a pathogenic agent,
(2) preparation of a template for the in vitro transcription of RNA with the aid of the cDNA library or part thereof, and
(3) in vitro transcription of the template.

The patient's tumour tissue can be obtained e.g. by a simple biopsy, but it can also be provided by the surgical removal of tumour-invaded tissue. Similarly, cells infected with a pathogenic agent (according to the invention, cells can of course also be invaded by several germs) can be obtained from biopsies of any infected tissue, e.g. the lymph nodes. Examples which may be mentioned of other particularly suitable sources of infected cells are blood, serum, lymph fluid and synovial fluid. Infected cells can optionally also be obtained from other body fluids such as sputum, sperm, vaginal fluid, urine, stool, sweat, etc. Also, the preparation of the cDNA library or part thereof according to step (1) of the preferred embodiment of the preparation method of the present invention can be carried out after the appropriate tissue or the cells have been deep-frozen for storage, preferably to temperatures below −70° C.

The first step in preparing the cDNA library or part thereof is to isolate the total RNA from the tumour tissue cells or the infected cells. Appropriate methods are described e.g. in Maniatis et al., op. cit. Appropriate kits are also commercially available, for example from Roche AG (e.g. the product “High Pure RNA Isolation Kit”). The appropriate poly(A⁺) NA is isolated from the total RNA by methods known to those skilled in the art (cf., for example, Maniatis at al., op. cit.). Appropriate kits are also commercially available. One example is the “High Pure RNA Tissue Kit” from Roche AG. The cDNA library is then prepared from the poly(A⁺) RNA obtained in this way (cf. also, for example, Maniatis at al., op. cit.). This step of the preparation of the cDNA library can also be carried out using commercial kits available to those skilled in the art, e.g. the “SMART PCR cDNA Synthesis Kit” from Clonetech Inc.

According to step (2) of the preferred embodiment of the preparation method described above, a template for the in vitro transcription is synthesized from the cDNA library (or part thereof). According to the invention, this is done particularly by cloning the resulting cDNA fragments into a suitable RNA production vector. The appropriate DNA template, and plasmids preferred according to the invention, have already been indicated above in connection with the preparation of the mRNA provided for transfection of the cells.

For in vitro transcription of the template prepared in step (2) above, said template, when it takes the form of circular plasmid (c)DNA, is first linearized with an appropriate restriction enzyme. Preferably, before the actual in vitro transcription, the construct cleaved in this way is purified once again using e.g. appropriate phenol/chloroform and/or chloroform/phenol/isoamyl alcohol mixtures. This ensures in particular that the DNA template is in protein-free form. The next step is enzymatic synthesis of the RNA from the purified template. This secondary step (substep) takes place in an appropriate reaction mixture containing the linearized, protein-free DNA template in a suitable buffer, to which a ribonuclease inhibitor is preferably added, using a mixture of the required ribonucleotide triphosphates (rATP, rCTP, rUTP and rGTP) and an adequate amount of an RNA polymerase, e.g. T7 polymerase. The reaction mixture here is in RNase-free water. Preferably, a CAP analogue is also added in the actual enzymatic synthesis of the RNA. After incubation at 37° C. for an appropriate time, e.g. 2 h, the DNA template is degraded by adding RNase-free DNase, incubation preferably being carried out again at 37° C.

Preferably, the RNA prepared in this way is precipitated with ammonium acetate/ethanol and optionally washed one or more times with RNase-free ethanol. Finally, the purified RNA is dried and, in a preferred embodiment, taken up in RNase free water. In addition, the RNA prepared in this way can be subjected to several extractions with phenol/chloroform or phenol/chloroform/isoamyl alcohol.

In another preferred embodiment of the present invention, only part of a total cDNA library is obtained and transferred into appropriate mRNA molecules. Therefore, according to the invention, it is also possible to use a so-called subtraction library as part of the total cDNA library in order to provide the mRNA molecules according to the invention. A preferred part of the cDNA library of the tumour tissue codes for the tumour-specific antigens. The appropriate antigens are known for certain tumours. In the case of cells infected with a pathogenic agent (or several different pathogenic agents), the cDNA partial library preferably codes for the products (polypeptides) of the (host) cell genes that are upregulated because of infection. In another preferred embodiment, the part of the cDNA library coding for the tumour-specific antigens, or the part of the cDNA library coding for the upregulated gene products, can be determined first (e.g. before step (1) of the method defined above). This is preferably done by establishing the sequences of the tumour-specific antigens, or of the gene products that are upregulated because of infection with the pathogenic agent, by matching with a corresponding MIA library from healthy tissue or uninfected cells.

The matching according to the invention comprises especially a comparison of the expression pattern of the healthy tissue with that of the tumour tissue in question, or, in the case of immune stimulation against a pathogenic germ, a comparison of the expression pattern of healthy cells with that of infected cells. Appropriate expression patterns can be determined at the nucleic acid level with the aid of suitable hybridization experiments, for example. This can be done e.g. by separating the appropriate (m)RNA or cDNA libraries of the tissues or cells in suitable agarose or polyacrylamide gels, transferring them to membranes and hybridizing them with appropriate nucleic acid probes, preferably oligonucleotide probes, which represent the respective genes (Northern or Southern blots). A comparison of the corresponding hybridizations thus provides the genes that are particularly suitable for immune stimulation, i.e. genes that are exclusively or more strongly expressed by the tumour tissue, or genes that are upregulated in infected cells.

In another preferred embodiment, said hybridization experiments are carried out with the aid of a diagnosis by means of microarrays (one or more microarrays). An appropriate DNA microarray comprises a definite arrangement, especially in a small or minimal space, of nucleic acid probes, especially oligonucleotide probes, each probe representing e.g. a gene whose presence or absence in the corresponding (m)RNA or cDNA library is to be investigated. Hundreds, thousands and even tens to hundreds of thousands of genes can thus be represented in an appropriate microarrangement. For analysis of the expression pattern of the particular tissue or particular cells, either the poly(A⁺) RNA or, preferably, the corresponding cDNA is then labelled with a suitable marker—fluorescent markers are used in particular for this put pose—and brought into contact with the microarray under suitable hybridization conditions. If a cDNA species binds to a probe molecule, especially an oligonucleotide probe molecule, present on the microarray, a more or less pronounced fluorescence signal is accordingly observed and can be measured with a suitable detection device, e.g. an appropriately designed fluorescence spectrometer. The more frequently the cDNA (or RNA) species is represented in the library, the more intense the signal, e.g. the fluorescence signal, will be. The corresponding microarray hybridization experiment or several or many such experiments) is (are) carried out separately for the tumour tissue and the healthy tissue or for the infected and uninfected cells. The difference between the signals read off in the microarray experiments therefore indicates the genes exclusively or more frequently expressed by the tumour tissue, or the genes that are upregulated because of infection with the pathogenic agent. Such DNA microarray analyses are described e.g. in Schena (2003), Microarray Analysis, ISBN 0-471-41443-3, John Wiley & Sons, Inc., New York, the relevant content of disclosure of said publication being fully included in the present invention.

However, the plotting of infection-specific or tumour tissue-specific expression patterns is in no way restricted to analyses at the nucleic acid level. Those skilled in the art are of course also familiar with methods known in the state of the art which are used for expression analysis at the protein level. The techniques of 2D gel electrophoresis and mass spectrometry may be mentioned in particular here, it also being advantageously possible for these techniques to be combined with protein biochips (i.e. microarrays at the protein level where e.g. a protein extract from healthy or tumour tissue or from an infected or uninfected cell is brought into contact with antibodies and/or peptides applied to the microarray substrate). As regards the mass spectroscopic methods, MALDI-TOF (matrix assisted laser desorption ionisation—time of flight) methods may be mentioned in particular. Said techniques of protein chemical analysis for obtaining the expression pattern of tumour tissue compared with healthy tissue or of infected cells compared with corresponding uninfected cells are described e.g. in Rehm (2000), Der Experimentator: Proteinbiochemie/Proteomics, Spektrum Akademischer Verlag, Heidelberg, 3rd ed., whose relevant content of disclosure is incorporated expressis verbis in the present invention. As far as protein microarrays are concerned, reference is made once again to the relevant details in Schena (2003), op. cit.

Apart from the transfected cells, the pharmaceutical composition according to the invention contains one or more pharmaceutically acceptable excipients and/or one or more pharmaceutically acceptable vehicles. Suitable excipients or vehicles are preferably sterile media or buffer solutions adapted to the particular blood cells.

Methods for the appropriate formulation and preparation of the pharmaceutical composition according to the invention are disclosed in “Remington's Pharmaceutical Sciences” (Mack Pub. Co., Easton, Pa., 1980), whose content is fully incorporated in the disclosure of the present invention. Examples of suitable excipients for parenteral administration, apart from sterile water, sterile buffer solutions or sterile media, are polyalkylene glycols, hydrogenated naphthalenes and, in particular, biocompatible lactide polymers, lactide/glycolide copolymer or polyoxyethylene/polyoxypropylene copolymers. Compositions according to the invention can contain fillers or substances, such as lactose or mannitol, substances for the covalent coupling of polymers, e.g. polyethylene glycol, substances for complexation with metal ions or the inclusion of materials in or on particular preparations of polymer compounds, e.g. polylactate, polyglycolic acid or hydrogel, or on liposomes, microemulsions, micelles, unilamellar or multilamellar vesicles, erythrocyte fragments or spheroplasts. The particular embodiments of the compositions are chosen as a function of the physical behaviour, for example in respect of solubility, stability, bioavailability or degradability. Controlled or constant release of the active components according to the invention in the composition entails formulations based on lipophilic depots (e.g. fatty acids, waxes or oils). Coatings of substances according to the invention, or compositions containing such substances, i.e. coatings with polymers (e.g. polyoxamers or polyoxamines), are also disclosed within the framework of the present invention. Furthermore, substances or compositions according to the invention can have protective coatings, e.g. protease inhibitors or permeability enhancers. Preferred aqueous excipients are e.g. water for injection (WFI) or water buffered with phosphate, citrate or acetate, etc., or appropriate cellular media, the pH typically being adjusted to between 5.0 and 8.0, preferably to between 6.0 and 7.0. The excipient(s) or vehicle(s) will preferably also contain salt constituents, e.g. sodium chloride or potassium chloride, or other components that render the solution isotonic, for example. The excipient(s) or the vehicle(s) can also contain, apart from the abovementioned constituents, additional components such as foetal calf serum, growth factors, human serum albumin (HSA), polysorbate 80, sugars or amino acids.

The method of administration and the dosage of the pharmaceutical composition according to the invention depend on the disease to be treated and how advanced it is, as well as on the patient's body weight, age and sex.

The concentration of the transfected cells in such formulations can therefore vary within wide limits, e.g. from 5×10⁴ to 1×10⁸ cells/ml. The pharmaceutical composition according to the invention is preferably administered to the patient parenterally, e.g. by the intravenous, intraarterial, subcutaneous or intramuscular route. The pharmaceutical composition of the present invention can also be injected locally, e.g. into a tumour.

Thus the invention also provides a method of treatment or a method of inoculation for the prevention of carcinoses or infectious diseases, e.g. the diseases mentioned above, which comprises administering the pharmaceutical composition according to the invention to a patient, especially a human.

In one preferred embodiment of the method of treatment or inoculation, or for the above-defined use of the blood cells transfected according to the invention, i.e. a mixture of red blood cells, granulocytes, mononuclear cells and/or blood platelets, or an enriched or substantially pure partial population thereof, for the preparation of a pharmaceutical composition for the treatment and/or prevention of carcinoses or infectious diseases, one or more cytokines and/or one or more co-stimulating molecules are administered to the patient in addition to the pharmaceutical composition according to the invention. As regards particularly suitable species administered in addition, cf. the above remarks pertaining to the molecules also encoded by the mRNA in addition to the antigen(s).

The invention therefore also generally provides a method of treatment or inoculation which comprises administering the blood cells transfected according to the invention, and at least one cytokine, e.g. one or more of the abovementioned cytokines, especially GM-CSF, to a patient, especially a human. The method is used especially for the treatment and/or prevention of appropriate carcinoses (e.g. the above carcinoses) or infectious diseases. Accordingly, the present invention further relates in general to a pharmaceutical composition comprising transfected blood cells (as defined above) and at least one cytokine, e.g. one or more of the abovementioned cytokines, such as GM-CSF, preferably in combination with a pharmaceutically acceptable excipient and/or vehicle. The invention thus also discloses the use of cytokines, e.g. one or more of the abovementioned cytokines, especially GM-CSF, in combination with the transfected blood cells as defined above, for the treatment and/or prevention of carcinoses (e.g. the carcinoses listed above) or infectious diseases (e.g. the infectious diseases listed above).

In another preferred embodiment of the present invention, the cytokine, e.g. GM-CSF, is administered simultaneously with or, preferably, before or after the pharmaceutical composition containing the cells transfected according to the invention (or is used for the preparation of a corresponding medicament for administration simultaneously with or before or after the blood cells listed above). Very particularly preferably, the cytokine, especially GM-CSF, is administered a short time (e.g. about 2 h or less, for instance up to about 5 min) before or a shorter time (e.g. about 5, 10, 15, 30, 45 or 60 min) or a longer time (e.g. about 2, 6, 12, 24 or 36 h) after administration of the pharmaceutical composition defined above, or generally after the cells transfected according to the invention.

The cytokine, e.g. GM-CSF, can be administered by the same route as the pharmaceutical compositions according to the invention or the blood cells transfected according to the invention, or by a different route. Suitable routes of administration, as well as possible suitable formulations in respect of the cytokine(s), can be found in the above remarks relating to the pharmaceutical compositions according to the invention. In a human patient, a GM-CSF dose of 100 micrograms/m² is particularly recommended. Particularly preferably, the cytokine, e.g. GM-CSF, is administered by s.c. injection.

Preferably, the pharmaceutical compositions of the present invention or the blood cells transfected according to the invention, and the cytokine(s) or other co-stimulating molecules optionally associated therewith, are administered in the form of interval doses. For example, a dose of a pharmaceutical composition according to the invention can be administered in shorter intervals, e.g. daily, every other day, every third day, etc., or, preferably, in longer intervals, e.g. once a week, once every two weeks, once every three weeks, once a month, etc. The intervals can also be variable here, it being necessary in particular to take account of the patient's immunological parameters. For example, the administration of a pharmaceutical composition according to the invention (and also the administration of the cytokine(s) or co-stimulating molecule(s) optionally associated therewith) can follow a treatment scheme in which the interval at the start of the treatment is shorter, e.g. once every two weeks, after which the interval is extended e.g. to once a month, depending on the course of treatment or the appropriately determined immunological parameters of the patient. Depending on the patient, especially his condition and immunological parameters, it is thus possible to apply a therapeutic scheme tailored to the particular individual.

NOM Overall, the invention therefore also provides a method of treatment in which blood cells or haemopoietic cells (as defined above) are first collected from a patient (animal or human), transfected in vitro according to the invention with the above-defined mRNA, and finally administered to an appropriate patient, preferably the same patient from whom the particular blood cells were withdrawn in the first step, account being taken of the relevant remarks regarding the formulation and administration of the pharmaceutical composition according to the invention.

Thus, according to the invention, blood cells or haemopoietic cells transfected with at least one mRNA comprising at least one region coding for at least one antigen are used to stimulate an immune response to the antigen(s) encoded by the mRNA (or are used to prepare a corresponding drug for immune stimulation).

Surprisingly, it has therefore been found according to the invention that it is not necessary, when vaccinating against certain antigens, to differentiate suitable blood cells into antigen presenting cells (APCs), especially dendritic cells (DCs), by means of expensive cell culture techniques, before transfection with an mRNA coding for the particular antigen, in order to trigger an appropriate immune response in the patient. APCs are distinguished in particular in that they interact with lymphocytes through the expression of co-stimulating molecules and the secretion of cytokines and are able to trigger an antigen-specific immune response via said lymphocytes. Other APCs apart from DCs are macrophages and B lymphocytes, Blood cells used according to the invention contain B cells, monocytes, T lymphocytes, optionally granulocytes and a small number of DCs, which can be divided into plasmacytoid DCs (pDCs) and myeloid DCs (mDCs). Without being bound to a particular theory of the mode of action of blood cells transfected in this way, it is assumed according to the invention, in the light of current knowledge, that the transfected cells administered to the patient, e.g. by injection, express the protein encoded by the mRNA and stimulate an antigen-specific immunity either directly (via the APCs present in the blood cells, such as the PBMCs) or indirectly (via the transfected cells, which are not APCs but which die and are absorbed by means of phagocytosis by APCs present in the organism, or by proteins secreted by the transfected cells, which are absorbed by means of phagocytosis by APCs present in the organism). Contrary to the state of the art, the present invention thus requires no expensive process steps, e.g. cell culture steps and the like, in order to obtain enriched APC populations or prepare artificial APCs, for example, in another way. Furthermore, cytokines do not have to be used in large amounts. Typically, according to the invention, the period between collection of the blood cells, e.g. blood withdrawal, and administration of the pharmaceutical composition used e.g. as a tumour or infection vaccine is only one hour to a few hours, e.g. 2 hours. According to the invention, in contrast to methods known hitherto, blood cells, generally a mixture of red blood cells, mononuclear cells, granulocytes and/or blood platelets, or enriched or substantially pure populations of these blood cells, demonstrate the requisite and desired immunocompetency, and are administered rather than APCs generated in vitro, affording the advantages listed above.

### BRIEF DESCRIPTION OF THE DRAWINGS

In the Figures:

FIG. 1 shows a graphical representation of the results of FACS experiments on the expression of EGFP in CD4-specific T helper cells or CD19-specific B cells. mRNA coding for EGFP (left graphs) or mRNA coding for the influenza matrix protein (control; right graphs) was transfected in vitro into fresh human PBMCs by electroporation. The expression of EGFP was then studied by means of FACS on the basis of the fluorescence of the EGFP, CD4-positive or CD19-positive cells were detected by means of fluorescent anti-CD4 antibodies (top graphs) or anti-CD19 antibodies (bottom graphs). The results show that some of the CD4-positive cells present in the PBMCs have taken up the mRNA and express EGFP.

FIG. 2 also shows graphical representations of the results of FACS experiments, which prove that PEMCs transfected with mRNA are capable of activating T cells against antigens encoded by the mRNA. The experiments were carried out for 2 different electroporation methods, on the one hand with the Nucleofector apparatus from AMAXA and on the other hand with the EASYJECT PLUS standard electroporation system from Equibio. Fresh human PBMCs were transfected either with EGFP mRNA or with an mRNA coding for the influenza matrix protein. PBMCs loaded with the GILGFVFPL peptide of the influenza matrix protein were used as the positive control. After transfection, or after loading in the case of the positive control, the cells were co-cultivated for one week with fresh autologous PBMCs. The cells were then labelled with PercP-labelled CD8 specific monoclonal antibodies (for detecting cytotoxic T cells) and HC tetramers that fluoresce due to PB labelling (which exclusively recognize cytotoxic T cells that are specific for the influenza matrix protein epitope presented by HLA-A*0201). In both electroporation methods, it is found that only 0.1 or 0.7% of the CD8-positive cytotoxic T cells are specific for the dominant influenza matrix protein epitope when the PBMCs used for stimulation have been transfected with EGFP mRNA. By contrast, in the case of the transfection of PBMCs with the mRNA coding for the influenza matrix protein, the observed frequency of CD8-positive cytotoxic T cells that are specific for the dominant influenza matrix protein epitope is 1.6% or 2.5% after one week. It can therefore be established according to the invention that the proliferation of cytotoxic T cells specific for the dominant influenza matrix protein epitope is induced by the electroporation of PBMCs with mRNA coding for the influenza matrix protein, but not by transfection with EGFP mRNA. This result is furthermore independent of the type of electroporation.

### DETAILED DESCRIPTION OF THE INVENTION

The present invention is illustrated in greater detail by the non-limiting Examples which follow.

### Example 1

### Isolation of PBMCs

Peripheral blood mononuclear cells were isolated from healthy HLA-A0201-positive donors. Mononuclear cells were obtained by Ficoll-Hypaque gradient centrifugation. The PBMCs obtained were washed three times with PBS.

### Example 2

### Transfection of PBMCs by Electroporation

The PBMCs obtained were transfected using the Nucleofector apparatus and the Human B-Cell Nucleofector Kit (both from AMAXA GmbH, Cologne, Germany) according to the manufacturer's instructions. 4×10⁶ cells were transfected with 5 micrograms of RNA per transfection.

The monoclonal antibodies CD4-PerCP and CD19-PE (both from BD Pharmingen) were used for the immune phenotyping of PBMCs transfected with the EGFP mRNA.

After transfection, 1.5×10⁶ cells were incubated to maturity in 24 well culture dishes (Greiner) in 1.5 ml of X-Vivo 15 medium (Bio Whittaker, Belgium) containing 100 mg/ml of LIPS (Sigma, Deisenhofen, Germany) and 2.5 mg/ml of TNF-α (R&D Systems). As the positive control, non-transfected mature PBMCs were loaded for 1 h with 1 mg/ml of the HLA-A*0201-restricted peptide (GILGFVFTL) of the influenza matrix protein. After incubation for 24 h, the mature PBMCs were washed with medium. The cells were then used to stimulate syngenic thawed PBMCs (1 cells per well). Fresh medium, 10 U/ml of recombinant IL-2 and 5 mg/ml of recombinant IL-7 (both from R&D Systems) were added after 4 days. After 6 days of culture the cells were labelled with a PE-labelled human HLA-A*0201 tetramer specific for the influenza matrix protein. CD8-specific cells were detected using an FITC-labelled anti-CD4 antibody directed against a PercP-labelled CD8 antibody.

### Example 3

### Expression of EGFP in Human PBMCs Transfected In Vitro

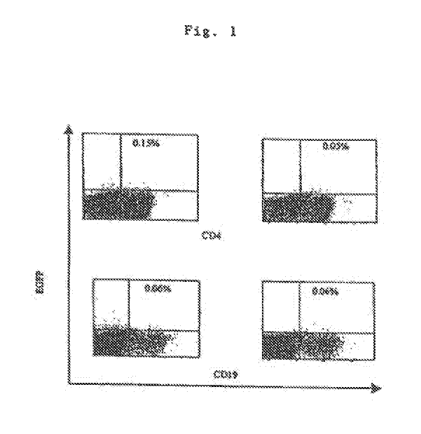

mRNA coding for EGFP Was transfected into fresh human PBMCs by electroporation (or lipofection, data not shown). One day after transfection the expression of EGFP in cells labelled with fluorescent monoclonal antibodies (anti-CD4 for T helper cells or anti-CD19 for B cells) was studied by FACS analysis. As shown in FIG. 1, some CD4-positive cells have taken up the mRNA and expressed EGFP. In some cases an expression of EGFP could also be found in E cells as well as in other cells that are not B or T cells, e.g. monocytes (not shown).

### Example 4

PEMCs Transfected with mRNA Activate T Cells

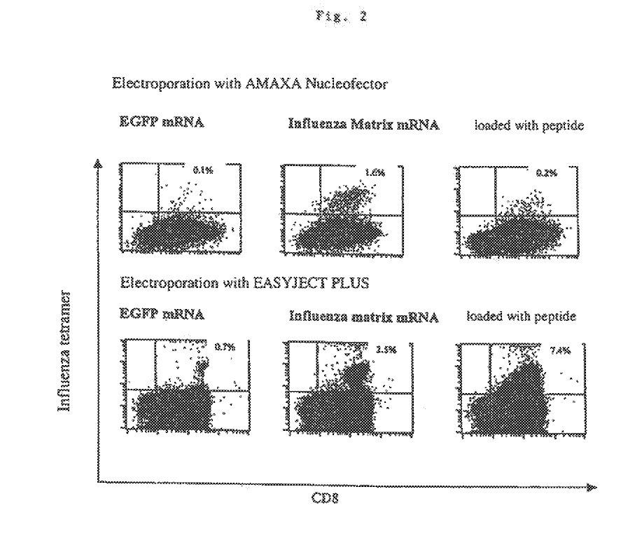

PEMCs from HLA-A*0201-positive healthy donors were transfected with mRNA coding for the influenza matrix protein. The transfections were performed using the Nucleofector apparatus from AMAXA or a standard electroporation system (EASYJECT PLUS) from Equibio. The transfected PBMCs were co-cultivated in vitro for one week together with autologous fresh PBMCs, a maturation optionally being carried out overnight by incubation with LPS and TNF-α. However, the same results were also obtained with non-stimulated transfected PBMCs. The cells were then labelled with monoclonal antibodies (anti-CD8 for CD8-positive cytotoxic T cells) and fluorescent MHC tetramers, the latter exclusively recognizing cytotoxic T cells that are specific for the influenza matrix protein epitope presented by HLA-A*0201. As shown by the results in FIG. 2, only the electroporation of mRNA coding for the influenza matrix protein induces a proliferation of cytotoxic T cells that are specific for the dominant epitope derived from the influenza matrix protein, whereas the transfection of EGFP mRNA does not cause any corresponding proliferation of CD8-positive cytotoxic T cells. This result is moreover independent of the electroporation system used and also independent of whether or not the transfected PBMCs were treated overnight with LPS and TNF-a before co-cultivation.

It can further be shown by means of in vivo experiments in mice that fresh splenocytes transfected by electroporation, as blood cells in terms in the present invention, are capable of triggering an immune response. This also applies especially to bicistronic mRNA coding for the antigen of interest and at the same time for a cytokine, a co-stimulating receptor, a homing molecule and/or a suicide molecule (which triggers necrosis or apoptosis).

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. Pharmaceutical composition comprising blood cells from whole blood and transfected with at least one mRNA, in combination with a pharmaceutically acceptable excipient and/for vehicle, the mRNA comprising at least one region coding for at least one antigen.

2-4. (canceled)

5. Pharmaceutical composition according to claim 1 wherein the mRNA comprises a region coding for at least one antigen from a tumour or a pathogenic agent.

6. Pharmaceutical composition according to claim 5 wherein the antigen(s) is/are a polyepitope of antigens from a tumour or a pathogenic agent.

7. (canceled)

8. Pharmaceutical composition according to claim 5 wherein the blood cells are transfected with a plurality of mRNA molecules that represent a cDNA library, or part thereof, of a tumour tissue or of a cell infected with a pathogenic agent.

9. (canceled)

10. Pharmaceutical composition according to claim 5 wherein the tumour antigen(s) is/are selected from the group consisting of 707-AP, AFP, ART-4, BAGE, β-catenin/m, Bcr-abl, CAMEL, CAP-1, CASP-8, CDC27/m, CDK4/m, CEA, CT, Cyp-B, DAM, ELF2M, ETV6-AML1, G250, GAGE, GnT-V, Gp100, HAGE, HER-2/neu, HLA-A*0201-R170I, HPV-E7, HSP70-2M, HAST-2, hTERT (or hTRT), iCE, KIAA0205, LAGE, LDLR/FUT, MAGE, MART-1/melan-A, MC1R, myosin/m, MUC1, MUM-1, -2, -3, NA88-A, NY-ESO-1, p190 minor bcr-abl, PmI/RARα, PRAME, PSA, PSM, RAGE, RU1 or RU2, SAGE, SART-1 or SART-3, TEL/AML1, TPI/m, TRP-1, TRP-2 TRP 2/INT2 and WT1.

11-12. (canceled)

13. Pharmaceutical composition according to claim 1 wherein the region coding for the antigen(s) and/or the 5′ and/or 3′-untranslated region of the mRNA is changed, relative to the wild-type mRNA, in such a way that it does not have any destabilizing sequence elements.

14. Pharmaceutical composition according to claim 1 wherein the mRNA contains a sequence region which is used to increase the translation rate.

15. Pharmaceutical composition according to claim 1 wherein the mRNA additionally codes for at least one cytokine and/or at least one co-stimulating molecule and/or at least one transcription factor and/or at least one homing receptor and/or at least one suicide gene.

16. Pharmaceutical composition according to claim 1 wherein the mRNA has a 5′ cap structure and/or a poly(A⁺) tail of at least about 25 nucleotides and/or at least one IRES and/or at least one 5′-stabilizing sequence and/or at least one 3′-stabilizing sequence.

17. (canceled)

18. Pharmaceutical composition according to claim 1 wherein the mRNA has at least one analogue of naturally occurring nucleotides.

19-20. (canceled)

21. Method of preparing a pharmaceutical composition according to claim 1, comprising the following steps:
(a) collection of blood cells, and
(b) transfection of the blood cells in vitro with at least one mRNA comprising at least one region coding for at least one antigen.

22. (canceled)

23. Method according to claim 21 wherein the period between the collection of blood cells in step (a) and the transfection in step (b) is less than 12 h, preferably less than 6 h and particularly preferably less than 2 h.

24-26. (canceled)

27. Method according to claim 21 wherein the mRNA for the transfection according to step (b) is complexed or condensed with at least one cationic or polycationic agent.

28. (canceled)

29. Method according to claim 21 wherein the mRNA is prepared by a method comprising the following steps:
(1) preparation of a cDNA library or part thereof from a patient's tumour tissue or from a patient's cells infected with a pathogenic agent,
(2) preparation of a template for the in vitro transcription of RNA with the aid of the cDNA library or part thereof, and
(3) in vitro transcription of the template.

30. Method according to claim 29 wherein the part of the cDNA library of the tumour tissue codes for the tumour-specific antigens or for the gene products that are upregulated because of infection with the pathogenic agent.

31. Method according to claim 30 in which the sequences of the tumour-specific antigens or the sequences of the gene products that are upregulated because of infection with the pathogenic agent are determined before step (1).

32. Method according to claim 31 wherein the determination of the sequences of the tumour-specific antigens or the sequences of the gene products that are upregulated because of infection with a pathogenic agent comprises matching with a cDNA library from healthy tissue or uninfected cells.

33. (canceled)

34. Pharmaceutical composition obtainable by the method according to claim 21.

35. A vaccine comprising a pharmaceutical composition of claim 34.

36. Method for stimulating an immune response to an antigen(s), particularly one(s) from a tumour or a pathogenic agent, comprising administering an immune response eliciting amount of a pharmaceutical composition, said pharmaceutical composition comprising blood cells transfected with at least one mRNA comprising at least one region coding for at least one of said antigen(s), optionally in combination with a pharmaceutically acceptable excipient and/or vehicle.

37-39. (canceled)

40. Method according to claim 35 wherein the immune stimulation is used for the therapy and/or prophylaxis of carcinoses or infectious diseases.
